(19) United States
(12) Patent Application Publication
(10) Pub. No.: US 2018/0008473 A1
(43) Pub. Date: Jan. 11, 2018

(54) BANDAGES

(71) Applicant: Anthony Leo Klein, Freeport, NY (US)

(72) Inventor: Anthony Leo Klein, Freeport, NY (US)

(21) Appl. No.: 15/714,859

(22) Filed: Sep. 25, 2017

Related U.S. Application Data
(63) Continuation-in-part of application No. 15/595,623, filed on May 15, 2017.
(60) Provisional application No. 62/336,526, filed on May 13, 2016.

(51) Int. Cl. A61F 13/02

(52) U.S. Cl. CPC A61F 13/023

(57) ABSTRACT

A bandage can be made oversized in width and length with a thick absorbent pad to provide pressure to the wound. The bandage provides a one-step procedure for wounds that need pressure and protection. The individually wrapped bandage is taken from a sterile wrapper to the wound so there is no worry of infection. Pull tabs make the bandage application a sterile procedure. Another bandage has a hard or semi hard cover with optional vent holes incorporated for air circulation. The bandage protects a wound or stitches or any delicate skin problem from being bumped or damaged from ordinary daily labors. The cover can have no pad in it or it can have a pad that can have nothing in it or can have anti-bacterial ointment, or the like, to help fight infection.

### CROSS-REFERENCE TO RELATED APPLICATIONS

This application is a continuation-in-part of U.S. patent application Ser. No. 15/595,623, filed May 15, 2017, which claims benefit of priority of U.S. provisional patent application No. 62/336,526, filed May 13, 2016, the contents of which are herein incorporated by reference.

### BACKGROUND OF THE INVENTION

### 1. Field of the Invention

One or more embodiments of the invention relates generally to bandages. More particularly, the invention relates to an adhesive bandage that either provide pressure to a wound site or provide a protective cover over a wound site.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Many types of bandages are available on the market. Conventionally, a bandage includes a wound contact area with an adhesive strip designed to attach the wound contact area to the wound.

Typically, bandages are designed with prints or in solid colors and are usually rectangular in shape, although other shapes are also within the conventional bandage arts.

In some circumstances, medical practitioners do not have a suitable bandage and make up their own. For example, when getting blood withdrawn, or after an IV is removed, the practitioner may place a wad of gauze, often formed by folding a 4 by 4 gauze several times, over the wound to apply pressure while taping the wadded gauze to the user. Several problems are realized with this practice. First, by wadding the gauze, even with a practitioner's gloved hand, the gauze is no longer sterile and, when placed directly on the point of needle puncture, could be a source for infection. Also, many times, a practitioner will ask a patient to hold the wadded gauze in place, while tape is recovered to tape the wadded gauze to the patient. This is another source of infection—from the patient's fingers. Finally, the tape itself is usually a roll that sits next to the practitioner's workplace and is not kept in a sterile environment.

Also, conventional bandages usually are typically placed directly over a wound. Should the user bump the wound, pressure applied to the outside of the bandage translates directly to pressure to the wound. This can slow or reverse healing of the wound. Some bandages provide a rigid dome over a wound, but these domes can completely seal over the wound, preventing air and moisture exchange at and near the wound site, often time slowing wound healing.

As can be seen, there is a need for bandages that address the various limitations of conventional bandages as discussed above.

### SUMMARY OF THE INVENTION

Embodiments of the present invention provide a bandage comprising a pad; and an adhesive strip extending from opposite ends of the adhesive strip, wherein the pad has a thickness defined between a wound application surface and an outer surface thereof; the adhesive strip extends from the outer surface of the pad; and the thickness is from about ¼ inch to about 1 inch.

Embodiments of the present invention further provide a method for applying pressure and protecting an injection site comprising removing a cover from a bandage to expose a pad and adhesive strips; applying pressure on a top surface of the pad onto the injection site; securing the adhesive strips to skin of a user, the adhesive strips extending from the top surface of the pad to provide pressure on the injection site by the pad when the adhesive strips are secured to the skin of the user, wherein the pad has a thickness defined between a wound application surface and the top surface thereof, the thickness being from about ¼ inch to about 1 inch.

Embodiments of the present invention also provide a method for protecting a wound site comprising applying a dome over the wound site, the dome creating a space between the wound site and an undersurface of the dome; adhering the dome to skin of a user with an adhesive strip.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 shows a detailed view of a bandage according to an exemplary embodiment of the present invention;

FIG. 2 shows a cross-sectional view of a first embodiment of a bandage for IV and/or blood testing, according to an exemplary embodiment of the present invention, in a wrapper-sealed configuration;

FIG. 3 shows a cross-sectional view of the bandage of FIG. 2, illustrating exposure of the sterile pad;

FIG. 4 shows a side view of the exposed sterile pad applied to a wound site prior to adhesively applying the adhesive strips;

FIG. 5 shows an alternative embodiment of disposing a protective wrapper over the bandage;

FIG. 6 shows the bandage of FIG. 2 applied to the user; and

FIG. 7 shows a bandage having a vented dome for covering a wound, according to another exemplary embodiment of the present invention, applied to a user.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

As is well known to those skilled in the art, many careful considerations and compromises typically must be made when designing for the optimal configuration of a commercial implementation of any item, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide medical bandages. One such bandage is oversized in width and length with a thick absorbent pad to give pressure to the wound. The bandage provides a one-step procedure for wounds from IV or needle puncture or other wounds that need pressure and protection. The provider does not need the assistance of the person being cared for or other people to help. The individually wrapped bandage is taken from a sterile wrapper to the wound so there is no worry of infection. Easy pull tabs make the bandage application a sterile procedure. Most providers currently use cotton and tape that has been in a drawer or on a table with no concern of bacteria or germs. Another bandage has a hard or semi hard cover with optional vent holes incorporated for air circulation. The bandage protects a wound or stitches or any delicate skin problem from being bumped or damaged from ordinary daily labors. The cover can have no pad in it or it can have a pad that can have nothing in it or can have anti-bacterial ointment, or the like, to help fight infection.

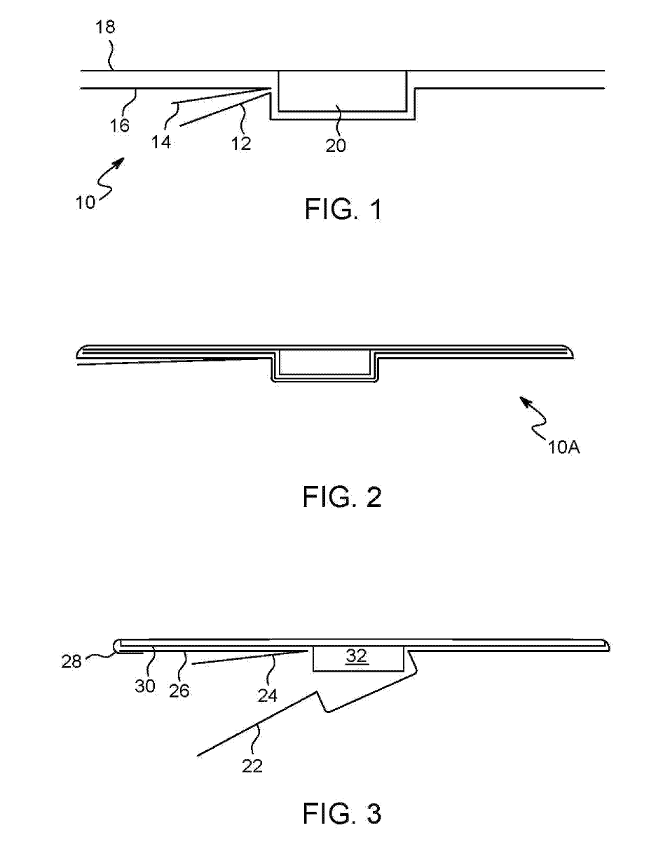

Referring to FIGS. 1 through 4, in some embodiments of the present invention, a bandage 10, 10A can be designed to cover injection sites, such as those created by an IV, a blood test, a blood donation, or the like. Bandages useful for these types of wounds would include a relatively thick gauze portion 20, 32 in the middle of the bandage with adhesive strips 18, 30 extending therefrom. The thick gauze portion may be from about ¼ to about 1 inch in thickness, for example, so that, when applied to the user, pressure is applied to the wound site.

The bandages may be individually wrapped so that sterility is ensured. The adhesive strips may be long enough to ensure hold and the application of pressure. For example, each of the adhesive strips may be from about 3 inches to about 6 inches in length. The bandages may be formed from a flexible material so that movement of the patient's arm would not cause the bandage to be removed or pull on the patient's skin.

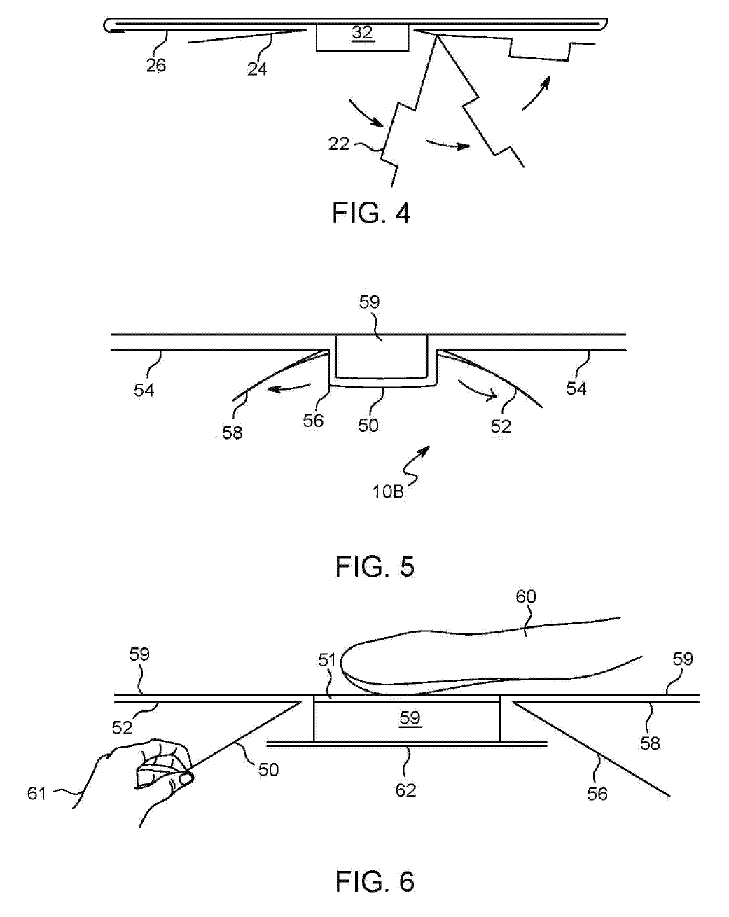

In some embodiments, as shown in FIG. 1, the bandage 10 may include a first pull tab 12 and a second pull tab 14 to remove a protective cover 16 from the pad 20 and the adhesive strip 18. In other embodiments, as shown in FIGS. 2 through 4, the bandage 10A can include a first pull tab 22 that covers the pad 32 and a portion of the adhesive 30, and a second pull tab 24 that may provide cover 26 over the adhesive 30. A link 28 between the two pull tabs 22, 24 may be disposed at one end of the adhesive 30 as shown in FIG. 3 so that pulling the first tab 22 completely off does not remove the second tab 24. In some embodiments, as shown in FIGS. 3 and 4, the first pull tab 22 may cover the pad 32 and fully encase both the adhesive side and the opposite side of the adhesive strip 30.

In some embodiments, as shown in FIG. 5, pull tabs 50, 56 may meet at a bottom part of the pad 59. Pulling the tabs 50, 56 may cause the cover 54 protecting the adhesive layer of the top strip to be removed.

FIG. 6 shows application of a bandage, such as bandage 10, 10A, 10B. A user may depress the pad 59 onto the skin 62 with one hand 60 after the pad 59 is exposed by moving the pull tabs 50, 56 with their other hand 61. The user may then continue to pull the tabs 50, 56 to remove the covers 52, 58 and reveal adhesive on the adhesive strip 59. The adhesive strip 59 may be adhered to the skin 62 such that pressure is applied to a wound via pad 59. Prior to the present invention, a user had to hold gauze on their wound site (such as an injection or blood draw site) or were wrapped with an elastic gauze to apply pressure to the wound. With the present invention, a single bandage can provide a sterile pad 59, with the appropriate pressure, to the wound site.

In some embodiments, as shown in FIG. 6, a support 51 may be disposed on a top surface of the pad 59. The support 51 may be formed, for example, from a rigid or semi-rigid plastic material that limits the flexibility of the pad 59 disposed there below. The support 51 may add pressure from the pad 59 to the skin 62 when the user attaches the adhesive strips 59 to the skin. In some embodiments, the adhesive strips may extend from a top portion of the support 51, as shown in FIG. 6.

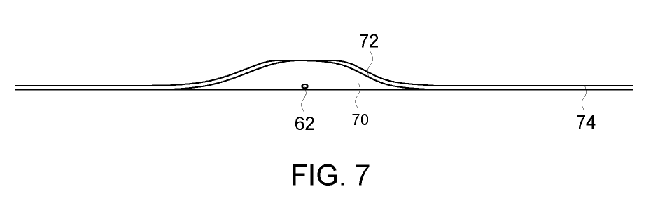

Referring to FIG. 7, in some embodiments of the present invention, a bandage can include a dome 72 that can be raised from a wound 62 to create a space 70 between the bandage and the wound 62. The dome 72 may be formed of a relatively rigid material so that pressure on the dome 72 does not cause it to collapse to contact the wound 62. The dome 72 may be vented to that air and moisture may be exchanged from under the dome to outside the dome. The dome may be padded around its perimeter/skin contact area. The dome may also include adhesive strips 74 to hold it in place at the appropriate location over the wound.

In some embodiments, the adhesive strips 74 may be integrally formed with the dome 72. In other embodiments, the dome 72 and adhesive strips 74 may be separate for individual application. The dome 72 may be designed in various sizes and shapes, such as round, oval, rectangular, or the like. The dome 72, for example, may be designed for small wounds, such as a small puncture wound, or for larger wounds, such as stitches, or the like.

In some embodiments, of the present invention, a bandage can include various designs and patterns. In some embodiments, the design may form the entire outer surface of the bandage. In some embodiments, the outer perimeter of the bandage would be cut out to match the design formed by the bandage. Of course, the design is formed on an outer surface of the bandage. The inner surface would include gauze and adhesive for adhering the bandage to the wound.

Along the same lines, the bandage could be formed in a three-dimensional shape. In other words, not only would the outer design of the bandage fully include the design, but the bandage surface may be raised, as appropriate, for the design.

The designs for the bandages can include flowers, sporting accessories, sport team logos, insects, cartoon characters, or the like. While conventional bandages have various designs printed on the outer surface, the present invention differs from these in that the entire outer shape and design of the bandage is the intended logo, character, print, or the like.

In some embodiments, the shape of the bandage may be formed like a tattoo, where another person may not be able to tell if the user is wearing a bandage or if they have a tattoo on their body. Such bandages could be formed in various designs. With this embodiment, the adhesive may be formed about an entire periphery of the gauze to ensure the bandage sits flush with the skin, without visible gaps between the bandage and the skin, thereby providing the illusion of a tattoo instead of a bandage.

In some embodiments, the bandages may be designed as a sun tattoo. In other words, the bandages may be formed so that, when the user's skin is in the sun, and the bandage is later removed, a sun tan tattoo is formed on the user. The sun tattoo may include both an external shape and internal cut-outs as necessary to achieve the desired effect. In this embodiment, the bandage may be formed from a flexible, reflective material. Moreover, the bandages falling in this embodiment of the present invention should be formed of a material that would not heat up its surface or the user's skin under the bandage, as the intention of such a bandage is to be used in the sun.

In each embodiment of the present invention, the bandages may include medication therein, such as antibacterial, anti-infective, anti-itch, acne treatment, or the like. In each embodiment of the present invention, the bandages may be individually wrapped to ensure sterility of the gauze.

In each embodiment of the present invention, the adhesive may be formed from an adhesive that can be easily removed from the skin.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A bandage comprising:
a pad; and
an adhesive strip extending from opposite ends of the adhesive strip, wherein
the pad has a thickness defined between a wound application surface and an outer surface thereof;
the adhesive strip extends from the outer surface of the pad; and
the thickness is from about ¼ inch to about 1 inch.

2. The bandage of claim 1, wherein the adhesive strips are from about three inches to about six inches in length.

3. The bandage of claim 1, further comprising a rigid or semi-rigid plastic support disposed at a top portion of the pad.

4. The bandage of claim 3, wherein the adhesive strip extends from the outer surface of the rigid or semi-rigid plastic support.

5. The bandage of claim 3, wherein the rigid or semi-rigid plastic pad has a support size and shape matching a pad size and shape of the pad.

6. A method for applying pressure and protecting an injection site, comprising:
removing a cover from a bandage to expose a pad and adhesive strips;
applying pressure on a top surface of the pad onto the injection site;
securing the adhesive strips to skin of a user, the adhesive strips extending from the top surface of the pad to provide pressure on the injection site by the pad when the adhesive strips are secured to the skin of the user, wherein
the pad has a thickness defined between a wound application surface and the top surface thereof, the thickness being from about ¼ inch to about 1 inch.

7. The method of claim 6, wherein the adhesive strips are from about three inches to about six inches in length.

8. The method of claim 6, further comprising a rigid or semi-rigid plastic support disposed at a top portion of the pad.

9. The method of claim 8, wherein the adhesive strip extends from the outer surface of the rigid or semi-rigid plastic support.

10. The method of claim 8, wherein the rigid or semi-rigid plastic pad has a support size and shape matching a pad size and shape of the pad.

11. A method for protecting a wound site comprising:
applying a dome over the wound site, the dome creating a space between the wound site and an undersurface of the dome;
adhering the dome to skin of a user with an adhesive strip.

12. The method of claim 11, wherein the adhesive strip is integral with the dome.

13. The method of claim 11, wherein the adhesive strip includes one or more strips attachable to the dome.

14. The method of claim 11, further comprising providing ventilation to the wound site via ventilation openings formed in the dome.

15. The method of claim 11, further comprising protecting the wound site from moisture by forming the dome as a solid structure contacting the skin of the user about its outer periphery.
